(19) United States
(12) Patent Application Publication
(10) Pub. No.: US 2024/0390070 A1
(43) Pub. Date: Nov. 28, 2024

(54) SYSTEM AND METHOD FOR COMPUTER-ASSISTED SURGERY

(71) Applicant: Kaliber Labs Inc., San Francisco, CA (US)

(72) Inventors: Chandra JONELAGADDA, San Francisco, CA (US); Aneesh JONELAGADDA, San Francisco, CA (US)

(21) Appl. No.: 18/693,945

(22) PCT Filed: Sep. 20, 2022
(86) PCT No.: PCT/US2022/076737

§ 371 Date: Mar. 20, 2024

Related U.S. Application Data
(60) Provisional application No. 63/246,050, filed on Sep. 20, 2021.

(51) Int. Cl. A61B 34/10; A61B 90/00

(52) U.S. Cl. CPC A61B 34/10; A61B 90/361; A61B 2034/107; A61B 2090/365

(57) ABSTRACT

Methods and apparatuses for modifying a video image (and/or a video stream) to include one or more virtual trajectory guides to assist a medical professional in performing a surgical procedure. These methods and apparatuses may include allowing the user to easily and quickly identify and/or select one or more regions (landmark regions), determine a surface contour of the identified landmark region, and generate a persistent virtual trajectory guide. Any of these methods may also include identifying (e.g., automatically identifying) one or more tools that may and guiding or assisting the user in manipulating the tools using the one or more virtual trajectory guide.

### CLAIM OF PRIORITY

This patent application claims priority to U.S. Provisional Patent Application No. 63/246,050, titled “SYSTEM AND METHOD FOR COMPUTER-ASSISTED SURGERY,” and filed on Sep. 20, 2021, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

In recent years, Artificial Intelligence has begun to be developed to be used to process images to recognize features of a human face as well as different anatomical structures in a human body. These AI tools can be used to automatically recognize an anatomical feature to assist an operator during a medical procedure. Computational methods such as machine learning and deep learning algorithms can be used for image or language processing to gather and process information generated in a medical procedure. The hope is to use AI algorithms that can then be used to or improve the outcome of the surgery. Current AI-assisted surgical systems and methods are still less than ideal in many respects to be used to, for example, guide a surgical procedure. Accordingly, improved AI-assisted surgical systems and methods are desired.

### SUMMARY OF THE DISCLOSURE

The methods and apparatuses (e.g., systems and devices, including software, hardware and/or firmware) for providing assistance in planning, analyzing and/or performing a surgery.

For example, described herein are computer-implemented method of assisting in a surgical procedure, the method comprising: receiving a video of a surgical site including an anatomical region; identifying, from user input, an arbitrary landmark on the anatomical region; identifying a hull contour conforming to a surface of the anatomical region that includes the arbitrary landmark; generating three-dimensional (3D) volumetric coordinates for a virtual trajectory guide extending from the hull contour; and outputting a modified video of the surgical site including the virtual trajectory guide, wherein an orientation of the virtual trajectory guide relative to the anatomical region is maintained as a field of view of the surgical site changes during the modified video.

In some examples a computer-implemented method of assisting in a surgical procedure includes: receiving a video of a surgical site including an anatomical region; identifying, from user input, an arbitrary landmark on the anatomical region; identifying a hull contour conforming to a surface of the anatomical region that includes the arbitrary landmark based on a distal tip region of a probe moved over the anatomical region; generating three-dimensional (3D) volumetric coordinates for a virtual trajectory guide extending from the hull contour using an axis normal to the hull contour; and outputting a modified video of the surgical site including the virtual trajectory guide, wherein the orientation of the virtual trajectory guide relative to the anatomical region is maintained as a field of view of the surgical site changes during the modified video.

Receiving the video of the surgical site may comprise capturing the video (e.g., video stream).

Identifying the arbitrary landmark may comprise extracting feature points from the anatomical region near the arbitrary landmark, further comprising excluding feature points that are on debris or rapidly-changing structures.

Identifying the hull contour may comprise identifying the hull contour based on a distal tip region of a probe moved over the anatomical region. For example, identifying the hull contour may include identifying a distal tip of the probe from the video and extracting 3D coordinates of the distal tip as it moves over the anatomical region.

Generating the 3D volumetric coordinates for the virtual trajectory guide may comprise generating the 3D volumetric coordinates so that the virtual trajectory guide passes through the arbitrary landmark.

The virtual trajectory guide may comprise a vector, a pipe, a cone, a line, etc. The appearance of the virtual trajectory guide may be adjusted or changed to indicate one or more properties of the virtual trajectory guide as described herein.

Any of these methods may include modifying the video of the surgical site to include the virtual trajectory guide, wherein the orientation of the virtual trajectory guide relative to the anatomical region is maintained as the field of view of the surgical site changes. For example, modifying the video may further comprise modifying the video to show the hull contour. In some examples, modifying the video further comprises modifying the video to show the arbitrary landmark.

Outputting the modified video may be performed in real time or near-real time.

Any of these methods may include identifying an instrument within the field of view of the video and comparing a trajectory of the instrument to the virtual trajectory guide. Any of these methods may include modifying the modified video to indicate that the trajectory of the instrument is congruent with the virtual trajectory guide above a threshold for congruence. The threshold for congruence may be user selected or predetermined. For example the threshold for congruence may be 50% or greater, 60% or greater, 70% or greater, 75% or greater, 80% or greater, 85% or greater, 90% or greater, 95% or greater, etc. In some examples the threshold for congruence is 75% or greater. Modifying the video may comprise changing an output parameter of the virtual trajectory guide. Identifying the instrument within the field of view of the video may comprise one or both of receiving user input of the instrument to be used or accessing a surgical plan to determining if the instrument is to be used.

Any of these methods may include modifying the virtual trajectory guide based on an instrument to be used during the surgical procedure.

Also described herein is software configured to perform any of these methods, e.g., non-transitory computer-readable medium including contents that are configured to cause one or more processors to perform the method. For example, a non-transitory computer-readable medium including contents that are configured to cause one or more processors to perform the method of: receiving a video of a surgical site including an anatomical region; identifying, from user input, an arbitrary landmark on the anatomical region; identifying a hull contour conforming to a surface of the anatomical region that includes the arbitrary landmark; generating three-dimensional (3D) volumetric coordinates for a virtual trajectory guide extending from the hull contour; and outputting a modified video of the surgical site including the virtual trajectory guide, wherein an orientation of the virtual trajectory guide relative to the anatomical region is maintained as a field of view of the surgical site changes during the modified video.

Also described herein are system configured to perform any of these methods. A system may be a system including one or more processors and a memory storing instructions to perform any of these methods. For example, a system may include: one or more processors; a memory coupled to the one or more processors, the memory storing computer-program instructions, that, when executed by the one or more processors, perform a computer-implemented method comprising: receiving a video of a surgical site including an anatomical region; identifying, from user input, an arbitrary landmark on the anatomical region; identifying a hull contour conforming to a surface of the anatomical region that includes the arbitrary landmark; generating three-dimensional (3D) volumetric coordinates for a virtual trajectory guide extending from the hull contour; and outputting a modified video of the surgical site including the virtual trajectory guide, wherein an orientation of the virtual trajectory guide relative to the anatomical region is maintained as a field of view of the surgical site changes during the modified video.

The inventions described herein may be used with the computer-assisted (AI assisted) surgical display techniques and systems described in each of the following international patent applications, herein incorporated by reference in their entirety: PCT/US2021/027109, titled “SYSTEM AND METHODS FOR AI-ASSISTED SURGERY,” filed on Apr. 13, 2021; PCT/US2021/027000, titled “SYSTEMS AND METHODS OF COMPUTER-ASSISTED LANDMARK OR FIDUCIAL PLACEMENT IN VIDEOS,” filed Apr. 13, 2021; and PCT/US2021/026986, titled “SYSTEMS AND METHODS FOR COMPUTER-ASSISTED SHAPE MEASUREMENTS IN VIDEO,” filed Apr. 13, 2021.

For example, described herein are computer-implemented method of assisting in a surgical procedure. Any of these methods may include: receiving a video of a surgical site including an anatomical region; identifying, from user input, an arbitrary landmark on the anatomical region; identifying a hull contour conforming to a surface of the anatomical region that includes the arbitrary landmark; generating three-dimensional (3D) volumetric coordinates for a virtual trajectory guide extending from the hull contour; and outputting a modified video of the surgical site including the virtual trajectory guide, wherein an orientation of the virtual trajectory guide relative to the anatomical region is maintained as a field of view of the surgical site changes during the modified video. Receiving the video of the surgical site may comprise capturing the video.

Identifying the arbitrary landmark may comprise extracting feature points from the anatomical region near the arbitrary landmark, further comprising excluding feature points that are on debris or rapidly-changing structures. Identifying the hull contour may comprise identifying the hull contour based on a distal tip region of a probe moved over the anatomical region.

Any of these methods may include identifying a distal tip of the probe from the video and extracting 3D coordinates of the distal tip as it moves over the anatomical region.

Generating the 3D volumetric coordinates for the virtual trajectory guide may comprise generating the 3D volumetric coordinates so that the virtual trajectory guide passes through the arbitrary landmark. The virtual trajectory guide may comprises a vector (e.g., arrow), line, pipe, etc.

Any of these methods may include modifying the video of the surgical site to include the virtual trajectory guide, wherein the orientation of the virtual trajectory guide relative to the anatomical region is maintained as the field of view of the surgical site changes. For example, modifying the video may further comprise modifying the video to show the hull contour. In some examples, modifying the video further comprises modifying the video to show the arbitrary landmark. Any of these methods may include outputting the modified video is performed in real time or near real-time.

Any of these methods may include identifying an instrument within the field of view of the video and comparing a trajectory of the instrument to the virtual trajectory guide.

In any of these methods and apparatuses, the modified video may be modified to indicate that the trajectory of the instrument is congruent with the virtual trajectory guide above a threshold for congruence. The threshold for congruence may be 50% or greater, 60% or greater, 70% or greater, 75% or greater, 80% or greater, 90% or greater, etc. (e.g., 75% or greater). Modifying the video may comprise changing an output parameter of the virtual trajectory guide. Identifying the instrument within the field of view of the video may comprise one or both of receiving user input of the instrument to be used or accessing a surgical plan to determining if the instrument is to be used. Any of these method may include modifying the virtual trajectory guide based on an instrument to be used during the surgical procedure.

For example, described herein are computer-implemented methods of assisting in a surgical procedure, the method comprising: receiving a video of a surgical site including an anatomical region; identifying, from user input, an arbitrary landmark on the anatomical region; identifying a hull contour conforming to a surface of the anatomical region that includes the arbitrary landmark based on a distal tip region of a probe moved over the anatomical region; generating three-dimensional (3D) volumetric coordinates for a virtual trajectory guide extending from the hull contour using an axis normal to the hull contour; and outputting a modified video of the surgical site including the virtual trajectory guide, wherein the orientation of the virtual trajectory guide relative to the anatomical region is maintained as a field of view of the surgical site changes during the modified video.

Also described herein are apparatuses, including device, system and software, for performing any of these methods. For example, described herein is software (e.g., non-transitory computer-readable medium including contents that are configured to cause one or more processors to perform any of these methods). In some examples, described herein is non-transitory computer-readable medium including contents that are configured to cause one or more processors to perform a method comprising: receiving a video of a surgical site including an anatomical region; identifying, from user input, an arbitrary landmark on the anatomical region; identifying a hull contour conforming to a surface of the anatomical region that includes the arbitrary landmark; generating three-dimensional (3D) volumetric coordinates for a virtual trajectory guide extending from the hull contour; and outputting a modified video of the surgical site including the virtual trajectory guide, wherein an orientation of the virtual trajectory guide relative to the anatomical region is maintained as a field of view of the surgical site changes during the modified video. As mentioned above, receiving the video of the surgical site may include capturing the video.

For example, any of these apparatuses may be configured to identify the arbitrary landmark may comprise extracting feature points from the anatomical region near the arbitrary landmark, further comprising excluding feature points that are on debris or rapidly-changing structures. Identifying the hull contour comprises identifying the hull contour based on a distal tip region of a probe moved over the anatomical region. Any of these apparatuses may be configured to identify a distal tip of the probe from the video and extracting 3D coordinates of the distal tip as it moves over the anatomical region.

Any of these apparatuses may be configured to generate the 3D volumetric coordinates for the virtual trajectory guide comprising generating the 3D volumetric coordinates so that the virtual trajectory guide passes through the arbitrary landmark. The virtual trajectory guide may comprises a vector, arrow, line, pipe, etc.).

These apparatuses may be configured to modify the video of the surgical site to include the virtual trajectory guide, wherein the orientation of the virtual trajectory guide relative to the anatomical region is maintained as the field of view of the surgical site changes.

In any of these apparatuses may be configured to modify the video by further modifying the video to show the hull contour. Modifying the video may further comprise modifying the video to show the arbitrary landmark. The apparatus may be configured to output the modified video is performed in real time.

Any of these apparatuses be configure to identify an instrument within the field of view of the video and compare a trajectory of the instrument to the virtual trajectory guide. The non-transitory computer-readable medium may further comprise modifying the modified video to indicate that the trajectory of the instrument is congruent with the virtual trajectory guide above a threshold for congruence.

As mentioned above, the threshold for congruence may be, e.g., 75% or greater. Modifying the video may comprise changing an output parameter of the virtual trajectory guide. Identifying the instrument within the field of view of the video may comprise one or both of receiving user input of the instrument to be used or accessing a surgical plan to determining if the instrument is to be used. The non-transitory computer-readable medium may further be configured to modify the virtual trajectory guide based on an instrument to be used during a surgical procedure.

Also described herein are systems configured to perform any of these methods. For example, a system may include: one or more processors; a memory coupled to the one or more processors, the memory storing computer-program instructions, that, when executed by the one or more processors, perform a computer-implemented method comprising: receiving a video of a surgical site including an anatomical region; identifying, from user input, an arbitrary landmark on the anatomical region; identifying a hull contour conforming to a surface of the anatomical region that includes the arbitrary landmark; generating three-dimensional (3D) volumetric coordinates for a virtual trajectory guide extending from the hull contour; and outputting a modified video of the surgical site including the virtual trajectory guide, wherein an orientation of the virtual trajectory guide relative to the anatomical region is maintained as a field of view of the surgical site changes during the modified video.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 is a flowchart representation of an example of a first system as described herein.

FIG. 2 is a schematic representation of an example of a method as described herein.

FIG. 3 is a schematic representation of an example of a method as described herein.

FIG. 4 is a schematic representation of an example of a method as described herein.

FIG. 5 schematically illustrates one example of a virtual trajectory guide engine that may be included as part of a controller of an apparatus as described herein.

FIGS. 6A-6C illustrate one example of frame of a view modified to include a virtual trajectory guide as described herein.

### DETAILED DESCRIPTION

Described herein are methods and apparatuses for modifying a video image (and/or a video stream) to include one or more virtual trajectory guides to assist a user, e.g., medical professional, surgeon, doctor, technician, etc., in performing a medical procedure, such as a surgical procedure. These methods and apparatuses may include allowing the user to easily and quickly identify and/or select one or more regions (landmark regions), determine a surface contour of the identified landmark region, and generate a persistent virtual trajectory guide. Any of these methods may also include identifying (e.g., automatically identifying) one or more tools that may and guiding or assisting the user in manipulating the tools (e.g., implants, manipulators, tissue modifying tools, etc.) using the one or more virtual trajectory guide. The virtual trajectory guide may be modified based on the procedure being performed and/or the tools detected. Thus, the three-dimensional (3D) shape of the structure on which the virtual trajectory guide is to be placed may be accurately and efficiently determined by tracing the location of a probe (e.g., in 3D space) using the video images. In particular, these methods may include estimating the general shape of the landmark area (e.g., using the probe) and identifying a convex and/or concave surface (e.g., “hull”) which best matches the contours of the surface of the landmark area. The virtual trajectory guide may then be posited at the desired location relative to the hull and movement of the virtual trajectory guide may be tracked as the hull (and the landmark area) moves relative to the camera or cameras.

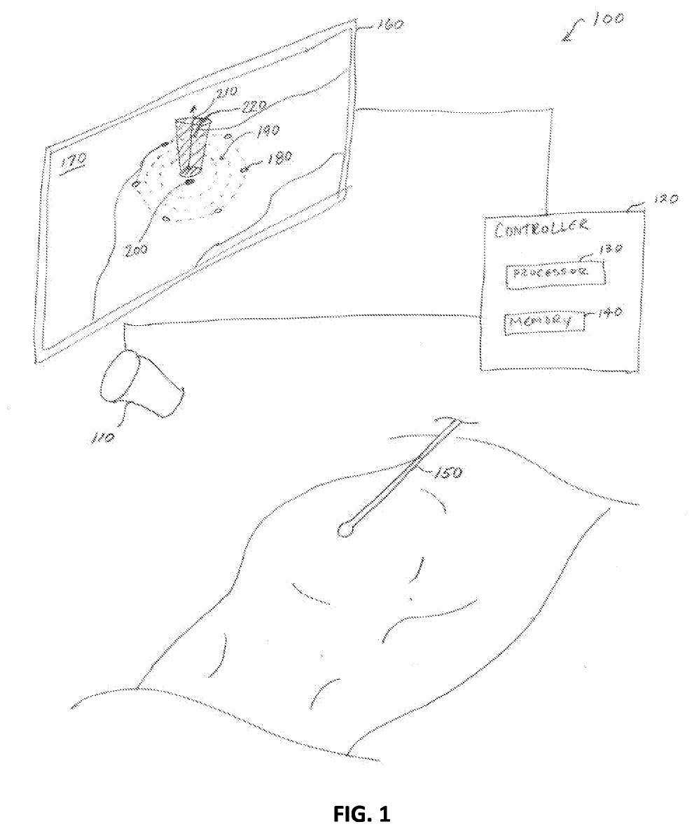

FIG. 1 shows an example of a system 100 for computer-assisted surgery that includes a camera 110 oriented at a surgical event and configured to capture a set of images (e.g., a series of still images or a video stream) of the surgical event. They system also includes a controller 120 connected or connectable to the camera 110 and configured to receive the set of images (or video stream) from the camera 110, locate a landmark location in the set of images in response to a landmark location of a probe 150, locate a three-dimensional location of the probe 150 in the set of images, generate a virtual landmark location in response to the landmark location of the probe, and generate a three-dimensional virtual hull about a surgical surface in response to the three-dimensional location of the probe 150. The controller 120 can further be configured to generate a virtual normal axis originating and/or intersecting with the virtual landmark location relative to the three-dimensional virtual hull and may generate a three-dimensional virtual trajectory guide in response to the virtual normal axis. The controller may include one or more processors and a variety of modules to assist in these processes.

As shown in FIG. 1, the system 100 can further include or be configured to output to a display 160 proximate the surgical event (and within a field of view of a surgeon) that may connect to the controller 120. The display 160 can: receive the output from the controller, such as renderings of the virtual landmark location 200 and the three-dimensional virtual trajectory guide 220; and render, augment, and/or overlay the virtual landmark location 200 and the three-dimensional virtual trajectory guide 220 in a second set of images captured by the camera 110 during a surgical procedure.

In some examples of the system 100 described in greater detail below, the camera 110 can be configured as or can include an endoscopic camera, and the display 160 can be an external monitor or set of monitors (e.g., high-definition LCD or LED monitors). In other variations of the system 100 described in detail below, the camera 110 can include a surgical theatre camera arranged with a field of view over an open surgical site and the display 160 can include an augmented reality vision system, such as a headset, goggles, glasses, etc.

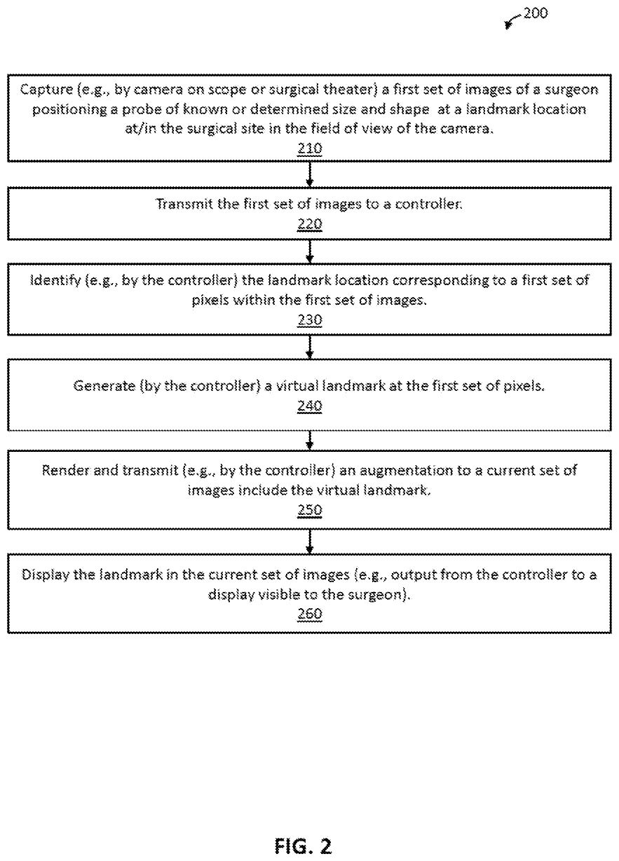

FIG. 2 illustrates one example of a method 200 (or portion of a method) for computer-assisted surgery as described herein. As shown in FIG. 2, this method may include capturing a first set of images of a surgeon positioning a probe of known size and shape at a landmark location at or in the surgical site in the field of view of the camera 210. The first set of images may be transmitted to a controller 220. The controller may identify the landmark location corresponding to a first set of pixels within the first set of images 230 and may generate a virtual landmark at the first set of pixels 240. The controller may then render and transmit an augmentation to a current set of images (e.g., being received by the camera, in real time), including the virtual landmark 250. The augmented images including the virtual landmark may then be sent for display (and/or storage) so that the landmark in the current set of images may be displayed to the surgeon 260.

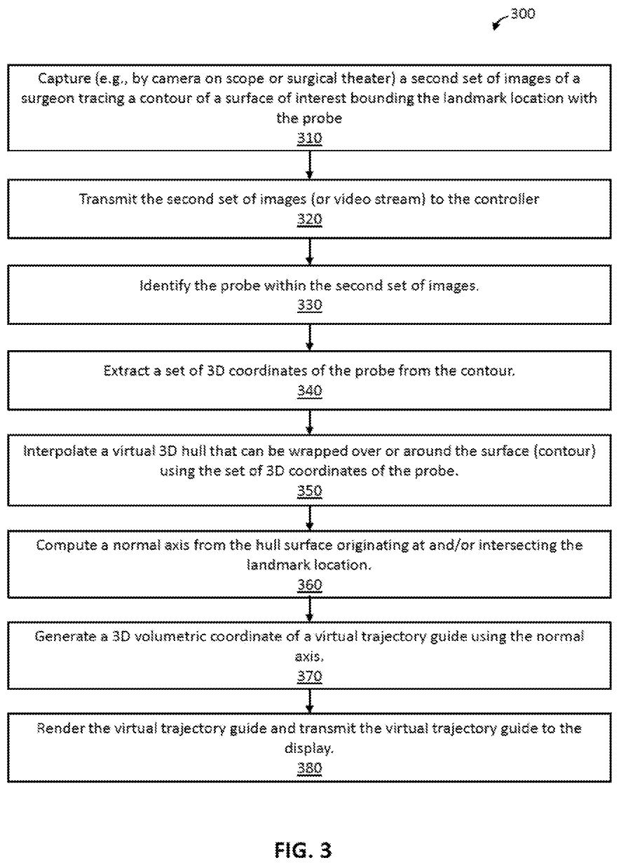

FIG. 3 shows another example of a method 300 (or portion of a method) for computer-assisted surgery. This method includes capturing a second set of images (using the camera) of a surgeon tracing a contour of a surface of interest bounding the landmark location with the probe 310. The camera may then transmit the second set of images to the controller 320. The controller may identify the probe within the second set of images 330. This method may also include extracting (e.g., in the controller) a set of three-dimensional (3D) coordinates of the probe from the contour 340. The controller may also interpolate a virtual three-dimensional hull that can be wrapped around the surface in response to the set of three-dimensional coordinates of the probe 350. The controller may then compute a normal axis from the hull surface originating at and/or intersecting the landmark location 360. The controller may also generate three-dimensional volumetric coordinates of a virtual trajectory guide in response to (e.g., using) the normal axis 370, and may render the virtual trajectory guide and transmit the virtual trajectory guide to the display 380.

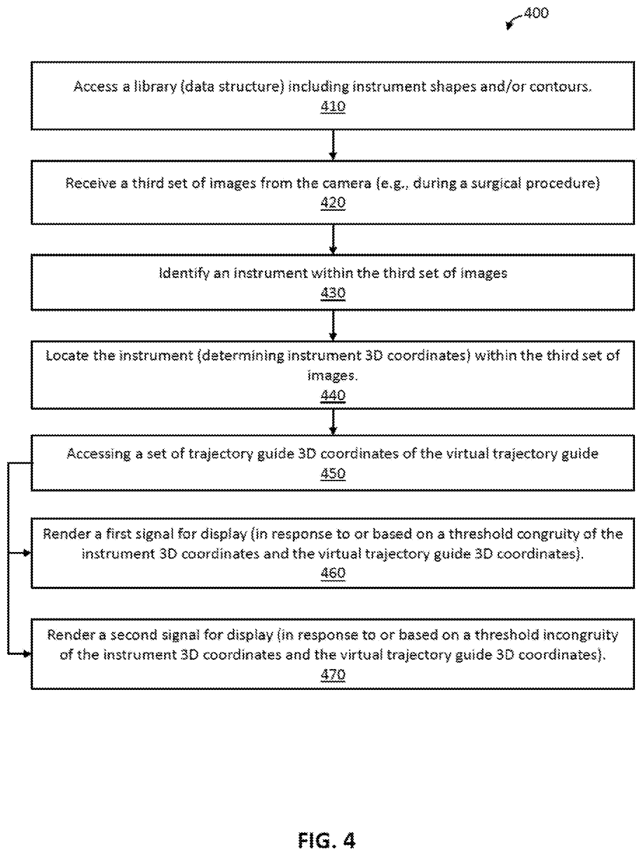

FIG. 4 shows another (e.g., third) method 400 or portion of a method for computer-assisted surgery that includes accessing an instrument shape and/or contour library (e.g., data structure) 410, and receiving a third set of images from the camera during a surgical procedure 420. The controller may then identify an instrument within the third set of images 430 and may locate the instrument in three-dimensional coordinates within the third set of images 440. This third method can further include accessing a set of trajectory guide three-dimensional coordinates of the virtual trajectory guide 450. The controller may then, in response to a threshold congruity of the instrument three-dimensional coordinates and the virtual trajectory guide three-dimensional coordinates, rendering a first signal for displaying by the display 460, and/or in response to a threshold incongruity of the instrument, three-dimensional coordinates and the virtual trajectory guide three-dimensional coordinates, may render a second signal for displaying by the display in Block 470.

Any of the apparatuses (e.g., systems and devices, including software, hardware and/or firmware) may perform all of some of the methods and their corresponding steps described above. For example, steps that automatically assist a surgeon (and/or surgical staff) in pre-surgical planning, surgical procedures, and post-surgical review. In particular, the apparatus (e.g., system 100) can implement the methods 200, 300, 400 described above to display a virtual landmark corresponding to a structure of interest on a display in the field of view of the surgeon, display a virtual trajectory guide for an instrument or implant placeable or implantable at the structure of interest on the display in the field of view of the surgeon, and, during the surgical procedure, generate and display real-time or near real-time guidance and/or feedback to the surgeon, displayed on the display in the field of view of the surgeon.

Generally, in a surgical procedure (e.g., arthroscopic, endoscopic, open), a surgeon and/or her surgical team visualize aspects of the surgery using a camera or a set of cameras configured to provide sets of still images or frames of video feeds. Surgeons also rely on physical landmarks, either naturally occurring within the patient's body or manually placed by the surgeon, to guide and navigate the camera and/or surgical instruments within the surgical site and ensure precise placement of instruments and/or implants. For example, surgeons typically use physical landmarks to keep track of latent vascularity, staple lines, suture locations, anatomical structures, and locations for affixing or implanting artificial structures or instruments.

Example of the system and methods described herein substantially reduce or eliminate excess cognitive load on the surgeon during a procedure by automatically identifying physical landmarks in response to a user input and generating a virtual landmark displayable and visible within a set of current surgical images on a display in the field of view of the surgeon. The display may be a high-definition monitor, set of high-definition monitors, and/or an augmented reality headset.

Furthermore, examples of the systems and methods described herein may further aid the surgeon by automatically identifying instrument (e.g., implant, screw, etcetera) trajectories in response to industry best practices, manufacturer specifications, and/or gold standard input from the surgical community and may generate a virtual trajectory guide for the instrument displayable and visible within a set of current surgical images on a display in the field of view of the surgeon.

Moreover, the examples of the systems and methods described herein may aid the surgeon and improve surgical outcomes by automatically identifying misaligned or altered instrument trajectories in response to a threshold incongruity between the actual position of the instrument during the surgical procedure and the virtual position of the virtual trajectory guide visible on the display. As described in detail below, these systems and methods can be configured to deliver a feedback signal indicative of a level of precision in the placement of the instrument, subject to an override by the surgeon, displayable and visible within a set of current surgical images on a display in the field of view of the surgeon.

Finally, these systems and methods described herein may improve the operation of the one or more processors (e.g., within the controller, or in one or more computer(s) performing as the controller). For example, the controllers described herein may include software instructions for performing these methods. In some cases, it is necessary that these methods be performed in real time or near real time (e.g., quickly), and therefore it is essential that they operate quickly and effectively. The combination and selection of the steps described herein, including the use of a variety of specific and custom automated agents (e.g., machine learning agents, deep learning agents, etc.) provide previously unrealized speed and accuracy even when operating in real time. The particular steps of these methods, the use of the specific automated agents, and the order in which these steps and agents are arranged, represent an improvement in the functioning of the processors and apparatuses performing these methods. These agents may be part of a module, which in turn may be part of an engine as described herein.

The examples of the systems and methods are described below with reference to example surgical procedures such as arthroscopic and endoscopic procedures. However, the example implementations of the system and methods are applicable to a range of surgical procedures, including for example: cardiac and/or cardiopulmonary surgery, neurosurgery, reconstructive surgery, radiotherapy, and/or robot-assisted surgery.

### Virtual Landmark

Generally, a system as described herein can assist a surgeon (and/or surgical staff) in identifying, marking, and non-transiently displaying a landmarked location at a surgical site during a surgical procedure (e.g., arthroscopic, endoscopic, open surgical procedures). As shown in FIG. 1 and FIG. 2, the systems described herein may include a camera that is configured and arranged to capture a first set of images of a surgeon positioning a probe of known size and shape at a landmark location at the surgical site in the field of view of the camera. For example, the camera can include an arthroscopic camera insertable adjacent a surgical site such as a joint (e.g., hip, shoulder, knee, elbow, etcetera). The camera can transmit the first set of images to a controller, which can include a processor 130 (or set of processors) and a memory 140. Generally, as used herein, the term set of images can include a single image, a discrete set of still images, or a continuous set of frames including images derived from a video camera operating at a frame rate.

The controller 120 can be configured to identify the landmark location corresponding to a first set of pixels within the first set of images. Generally, the controller 120 can identify a first set of pixels in an image from the first set of images corresponding with the probe 150 of known shape and size and can assign and/or register a set of coordinates to the first set of pixels. For example, the controller 120 can identify the probe 150 within the field of view of the camera 110 in an image, associate a portion of the size and shape of the probe 150 (e.g., a leading edge or tip of the probe) with a set of pixels within the image to the probe 150, and assign or register a set of coordinates to the location of the portion of the probe 150 within the field of view of the image.

Additionally or alternatively, the controller 120 can be configured to: receive or access a user input (e.g., from a surgeon or surgical assistant) identifying an image or set of images in which the probe is located at the location of interest in the field of view of the image or set of images. For example, the probe 150 can include a user interface such as a button or a microphone through which a user can transmit an input (e.g., tactile/manual or voice command). The probe 150 can be further configured to transmit the user input to the controller 120 such that the controller 120 can associate the user input with a time and location of the probe 150 (or portion of the probe 150) within the image or set of images.

In some examples the controller 120 can generate a virtual landmark at the first set of pixels. The controller 120 can generate the virtual landmark by associating and/or registering the first set of pixels with a location of interest, for example in response to a user input received from the probe 150 as noted above. The controller 120 can then execute Block S150 of the method S100 by rendering and transmitting an augmentation to a current set of images including the virtual landmark to a display 160. In one variation of the example implementation, the augmentation to the current set of images can include a coloration (e.g., directing the display 160 to display the virtual landmark in a clearly visible or distinguishable color(s)).

The system can further include a display arranged in a field of view of the surgeon (or surgical staff) and configured to display a current set of images. The display can display the virtual landmark in the current set of images for the surgeon to view within the field of view of the surgeon. In one example, the display can display the virtual landmark as a contiguous blue or cyan colored set of pixels arranged in a dot or circle that readily distinguishes the virtual landmark from the surrounding tissues in the surgical site including, for example, bone, cartilage, soft tissues, etcetera.

### Virtual Trajectory Guide

The systems and methods described herein may be configured to capture a second set of images of a surgeon (or surgical staff) tracing a contour of a surface of interest bounding the landmark location (and/or virtual landmark) with a probe. As noted above, the camera can include an arthroscopic camera insertable adjacent a surgical site such as a joint (e.g., hip, shoulder, knee, elbow, etcetera). Furthermore, as noted above, the probe can include a user interface (e.g., manual or voice input) that indicates and/or labels a set of motions of the probe as a tracing of a contour. Alternatively, the probe can include a set of probes that are uniquely configured for identifying landmark locations, tracing contours, etc.

In any of these methods and apparatuses, the camera can transmit the second set of images to the controller and the controller may identify the probe within the second set of images and extract a set of three-dimensional coordinates of the probe from the contour. In some examples the controller can, for each position in a set of positions along the contour, measure a number (N) of pixels in the second set of images representing the probe, from which the controller can infer a spatial relationship between the set of positions of the probe in three-dimensions. For example, the controller can select a set of three probe positions along the contour; measure a pixel count for each of the three positions (P1, P2, P3); and then calculate and/or triangulate a relative position of the probe and therefore a general three-dimensional set of coordinates for the set of three probe positions within the field of view of the surgical site. Additionally or alternatively, the controller can select a larger set of probe positions for example four, five, six, N-positions, along the contour from which the controller can calculate or triangulate the three-dimensional set of coordinates for the N-positions.

In any of these examples the controller 120 can execute Block S240 of the method S200 by: interpolating a virtual three-dimensional hull 190 wrappable around the surface in response to the set of three-dimensional coordinates of the probe 150 along the contour. Generally, the virtual three-dimensional hull 190 can be any three-dimensional geometry or manifold based, in part, upon the geometry of the underlying surface (e.g., surgical site) and its shape. For example, if the underlying surface is a substantially planar bony structure, then the virtual three-dimensional hull 190 can be substantially planar in geometry. Conversely, if the underlying surface is a substantially convex bony structure, then the virtual three-dimensional hull 190 can be substantially convex in geometry. Similarly, the virtual three-dimensional hull 190 can exhibit a generally concave geometry or a complex (e.g., partially concave, partially convex) geometry based upon the geometry of the underlying surface, the three-dimensional coordinates of the N-positions 180 along the contour, and the number N of points selected by the controller 120 in generating the virtual three-dimensional hull 190.

The controller can compute a normal axis from or through the surface of the virtual three-dimensional hull originating at and/or intersecting the virtual landmark. As described in detail below, the normal axis can function as a geometric reference for the controller in computing and/or generating a virtual trajectory guide. In some examples, the virtual three-dimensional hull can be rendered and displayed on the display augmenting or overlaid on a current set of images. Alternatively or additionally, the controller can render the virtual three-dimensional hull as a solid geometry (e.g., solid surface or structure); and, in response to an input from a user (e.g., surgeon, surgical assistant), direct the display to virtually rotate the virtual three-dimensional hull about the normal axis and/or the virtual landmark.

The controller can generate three dimensional (3D) coordinates of a virtual trajectory guide in response to the normal axis. The controller can render a virtual trajectory guide and transmit the virtual trajectory guide to the display for augmentation or overlay upon the current set of images. In one variation of the example implementation, the virtual trajectory guide can be a displayed set of pixels, similar to the virtual landmark, that readily permit a user to identify an optimal, suggested, or selected trajectory for an instrument to be used during the surgical procedure. In another variation of the example implementation, the virtual trajectory guide can be rendered by the controller and displayed by the display as a colorized line, pipe, post, or vector arranged with the virtual landmark to guide an approach angle and placement of an instrument at or within the surgical site. Alternatively, the virtual trajectory guide can be rendered by the controller and displayed by the display as a conical, cylindrical, or solid geometric (virtually three-dimensional solid) shape arranged with the virtual landmark to guide an approach angle and placement of an instrument at or within the surgical site.

In any of these methods and apparatuses, the controller can be configured to: access a surgical plan, identify a set of instruments (e.g., screws, anchors) selected within the surgical plan; and access a set of recommended practices for use of the set of instruments (e.g., surgical guidance, manufacturer specifications, etcetera). Based upon automated selection or user input, the controller can then: identify an instrument intended for use in a current surgical procedure; receive or ingest a set of geometric measurements of the selected instrument (e.g., length, body diameter, head diameter); and receive or ingest a recommended placement of the selected instrument (e.g., normal to the surface, offset from normal to the surface, etcetera). In this variation of the example implementation, the controller can then: render the virtual trajectory guide based upon the received or ingested geometry of the selected instrument and the received or ingested recommended placement of the selected instrument.

For example, an arthroscopic surgery plan may include the use of a screw with a body diameter between 2-3 millimeters that the manufacturer recommends be inserted at 30 degrees off-normal for optimal durability and functionality. Accordingly, the controller can render the virtual trajectory guide such that it includes a set of pixels representing a geometry equal to or greater than that of the instrument, for example such that the virtual trajectory guide 220, when displayed, is equal to or slightly larger in diameter (virtually 4-5 millimeters in diameter) than the instrument as displayed on the display. Therefore, when viewed by the surgeon (or surgical staff) during a surgical procedure, the instrument will appear to fit within the geometry of the virtual trajectory guide such that the surgeon can properly align the instrument with the surgical site during implantation.

Additionally, the controller can be configured to render the virtual trajectory guide at a 30-degree angle relative to the normal axis. Accordingly, the controller can receive or ingest a set of guideline measurements and parameters for the instrument and render the virtual trajectory guide such that, when displayed at the display, the surgeon (or surgical staff) will see the virtual trajectory guide augmenting the current set of images and guiding the instrument along a virtual trajectory (e.g., an inverted cone) at the appropriate or recommended angle of approach relative to the surface.

Additionally or alternatively, the system can be configured to accommodate displays having differing resolutions (e.g., differing number of pixels) available to display during surgery. Generally, if the overall number of pixels displayable by the display increases, then each pixel can represent a smaller increment in real space and thus virtually occupy a smaller measure of real space. Therefore, with increasing resolution of the display, the controller can be configured to revise and concentrate the number of pixels associated with the virtual trajectory guide such that an error bound is minimized for a given display resolution (e.g., HD, 4KUHD, etc.).

These methods and apparatuses may provide for real-time surgical guidance. For example, the controller can receive, and/or access a library of an instrument shape and/or contour, such as for example a size and/or shape of a surgical implant or screw to be used according to a surgical plan. In the example implementation, the controller can then receive a third set of images from the camera during a surgical procedure and identify the instrument within the third set of images based upon the library of instrument shape and/or contour. For example, the controller can receive the third set of images from the camera and identify the instrument within the third set of images by locating and tagging pixels in the third set of images that correspond to the shape and/or contour of the instrument.

In any of these examples the controller can locate the instrument in a set of instrument three-dimensional coordinates within the third set of images and access a set of trajectory guide three-dimensional coordinates of the virtual trajectory guide. Therefore, the controller can locate the instrument in three dimensions based upon the third set of images by implementing techniques described above with reference to the probe. For example, the controller can measure and/or detect a number of pixels in the third set of images that correspond to the instrument and, based upon the known geometry of the instrument, assign or register a set of three-dimensional coordinates to one or more points, features, or aspects of the instrument. For example, the controller can generate a multi-aspect coordinate set for a screw including: a screw tip coordinate set; a screw body coordinate set at or near a center of mass of the screw; and a screw head coordinate set at or near a center of a screw head. Generally, a set of coordinates associated with three aspects of an instrument can virtually locate the instrument relative to the virtual trajectory guide, although the controller can also compute coordinates based upon fewer or more aspects of the instrument.

In response to a threshold congruity of the instrument three-dimensional coordinates and the trajectory guide three-dimensional coordinates, the controller may render a first signal. In response to a threshold incongruity of the instrument three-dimensional coordinates and the trajectory guide three-dimensional coordinates, the controller may render a second signal. Additionally, the controller can direct the display to display the first signal or the second signal in real-time or near real-time during a surgical procedure.

Generally, the controller can interpolate a position of the instrument relative to the virtual trajectory guide by measuring a congruity of the coordinates of the respective bodies in real-time or near real-time. Therefore, if the controller determines a high level of congruity between the coordinates of the respective bodies (e.g., greater than 75 percent congruous), then the controller can render a first signal to be displayed at the display. For example, in a case of high congruity, the first signal can include causing the display to change a coloration of the pixels representing the virtual trajectory guide, for example from a blue or cyan color to a green color indicating to the surgeon that she has properly aligned the instrument with the virtual trajectory guide. On the contrary, in a case of low congruity (e.g., less than 75 percent congruous), the second signal can include causing the display to change a coloration of the pixels representing the virtual trajectory guide from a blue or cyan color to a red or yellow color indicating to the surgeon that she has improperly aligned the instrument with the virtual trajectory guide. In general, the threshold for congruence may be preset or user modified. For example, the threshold for congruence may be 50% or greater, 60% or greater, 70% or greater, 75% or greater, 80% or greater, 85% or greater, 90% or greater, 95% or greater, etc. or any percentage therebetween. In some cases the color and/or intensity (e.g., hue) of the virtual targeting guide may be adjusted based on the determined congruence (e.g., the more congruent, or higher the percent of congruence between the actual trajectory and the virtual trajectory guide, the darker or more intense the color may be).

In use, the controller can continuously receive images or streams of images from the camera and thus interpret, calculate, render, and direct first and second signals to the display in real-time or near real-time such that the surgeon obtains real-time or near real-time visual feedback on the alignment of the instrument with the surgical site prior to and/or during implantation. For example, if a surgeon moves the instrument during the procedure, then the camera can capture a set of images recording the movement of the instrument; the controller can receive the set of images and determine a threshold congruity or incongruity within the respective coordinate sets; the controller can render and transmit a first or second signal to the display and the display can display the first or second signal (e.g., green or red coloration of the virtual trajectory guide) to the surgeon. The system can implement the foregoing techniques in a continuous manner during a surgical procedure to generate and display visual feedback to the surgeon (and/or surgical team) during the surgical procedure.

Additional variations of the example implementations of the system and methods are described below may include verifying that a tool in the field of view is correct or alerting that it is not correct, dynamically maintain multiple virtual landmarks and virtual trajectory guides in multiple interconnected fields of view, etc.

In one variation of the methods and apparatuses described herein, the apparatus can be configured to recognize and/or warn a surgeon (or surgical staff) when an incorrect instrument is in use or within the field of view of the camera. For example, the controller can receive a third set of images from the camera in which a surgeon may mistakenly use an incorrect instrument (e.g., an incorrectly sized screw or implant); identify the incorrect instrument implementing the techniques and methods described above, emit a third signal that includes a warning to be displayed to the surgeon (or surgical staff), and transmit the third signal to the display. The display can then display the third signal (warning) to the surgeon. In one alternative to the variation of the example implementation, the system can accommodate a surgeon override through a user input (e.g., manually or voice-activated), upon receipt of which the controller can instruct the display to discontinue displaying the third signal.

For example, the controller can access or ingest a surgical plan that sets forth the use of a screw measuring 8 millimeters in length and 2 millimeters in diameter. During the surgical procedure, the controller can identify a screw measuring 12 millimeters in length and 3 millimeters in diameter in the third set of images; raster the third signal; and transmit the third signal to the display to warn the surgeon regarding the geometry of the screw. In response, the surgeon can either remove the selected screw from the field of view of the camera and retrieve the proper screw or, through manual or voice-activated input, override the system because the surgeon is making an adjustment to the surgical plan during the surgical procedure based upon her experience and judgement.

Similarly, the controller can access or ingest a surgical plan that sets forth the use of a screw with a recommended insertion angle of 70 degrees from normal (e.g., acute angle of approach). However, based upon experience and judgement, the surgeon may decide that a less acute angle of approach is better given the anatomy, age, mobility, or other condition of the patient. Therefore, during the surgical procedure, the controller can identify the screw in the third set of images, determine that the coordinates associated with the screw are incongruous with those of the virtual trajectory guide, generate the second signal and transmit the second signal to the display to colorize the virtual trajectory guide (e.g., a red or warning coloration). In response, the surgeon can, through manual or voice-activated input, override the system 100 because the surgeon is making an adjustment to the surgical plan during the surgical procedure based upon her experience and judgement. In response to the surgeon override, the controller can update the coordinates defining the virtual trajectory guide, generate an updated virtual trajectory guide 220 and direct the display to display the updated virtual trajectory guide such that it displays a congruous coloration in accordance with a first signal.

In one example the system can be configured to implement the foregoing techniques and methods to display a set of virtual trajectory guides in the display in response to a dynamic field of view of the camera. For example, a complex surgery can include a set of instruments or implants, each of which can be implanted at a different location corresponding to a unique landmark in the structure, (e.g., a set of screws to repair a broken bone or set of bones). Accordingly, the system can be configured to generate and store (e.g., in a memory) a successive set of virtual landmarks and virtual trajectory guides as the surgery progresses.

For example, the surgical plan may require a set of three screws placed in distal locations. Due to anatomical constraints or ease of access to the surgical site, the surgeon may elect to place a third screw first, a second screw second, and a first screw third. In this variation of the example implementation, the system can receive an input from a surgeon to locate a first virtual landmark at a first location at a first time in a first field of view of the camera, a second virtual landmark at a second location at a second time in a second field of view of the camera, and a third virtual landmark at a third location at a third time in a third field of view of the camera. The system can then implement techniques and methods described above to generate, raster, and display the respective virtual trajectory guides corresponding to each virtual landmark. Therefore, the system can generate and retain the respective virtual landmarks and virtual trajectory guides during the surgical procedure such that the surgeon has the option to select all three landmark locations serially and then, after identification of all three landmark locations, place or insert the instruments or implants at the landmark locations according to her best judgement.

The methods and apparatuses described herein may be used for minimally invasive and/or for open surgical procedures. In some examples the foregoing techniques and methods can be applied to open (e.g., non-arthroscopic and non-endoscopic) surgical procedures. As noted above, the system can be configured for open surgical procedures in which the camera can include a theatre camera or camera array arranged above a surgical procedure and the display can include an augmented reality/virtual reality (AR/VR) headset, goggles, or glasses configured to present a display in a field of view of the surgeon. For example, the camera can include a magnifying or telephoto lens that can zoom into a surgical site in order to populate the display with a set of pixels relating to the surgical site while excluding extraneous features within the field of view of the camera. Additionally, the display can include a set of fiducials registered to an external reference (e.g., the surgical theatre) such that the orientation and/or perspective of the display relative to the surgical site can be registered and maintained by the controller.

In another example, an open surgery can include a hip replacement surgery in which a surgeon replaces one or both of a hip socket in the pelvic bone and/or the head of the femur. In this variation of the example implementation, the system can identify, locate, raster (e.g. prepare), and display a set of virtual landmarks at the surgical site; generate, raster, and display a set of virtual trajectory guides at the virtual landmarks and implement real-time surgical guidance to the surgeon at her display during the surgical procedure.

For example, a surgeon can manipulate a probe in combination with a user input to identify a site within the hip socket requiring ablation, in response to which the controller can implement methods and techniques described above to raster (e.g., generate) a set of pixels representing the virtual landmark, generate a three-dimensional virtual hull, generate a virtual normal axis originating and/or intersecting with the virtual landmark location, and generate a three-dimensional virtual trajectory guide in response to the virtual normal axis. The controller can transmit the foregoing renderings to the display (e.g., AR headset) so that the surgeon can position, align, and apply an ablation tool to the hip socket to remove excess bone and tissue. Furthermore, the controller and display can implement surgical guidance techniques and methods described above to generate, raster, transmit, and display feedback signals to the surgeon during the ablation procedure to ensure proper alignment and depth of ablation of the hip socket. Additionally, the system can implement the techniques and methods described above to serve visual guidance and feedback during alignment and placement of the replacement hip socket and associated implants.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor, but any suitable dedicated hardware device can (alternatively or additionally) execute the instructions.

The methods and apparatuses described herein, including in particular the controllers described herein, may include one or more engines and datastores. A computer system can be implemented as an engine, as part of an engine or through multiple engines. As used herein, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine's functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein.

The engines described herein, or the engines through which the systems and devices described herein can be implemented, can be cloud-based engines. As used herein, a cloud-based engine is an engine that can run applications and/or functionalities using a cloud-based computing system. All or portions of the applications and/or functionalities can be distributed across multiple computing devices and need not be restricted to only one computing device. In some embodiments, the cloud-based engines can execute functionalities and/or modules that end users access through a web browser or container application without having the functionalities and/or modules installed locally on the end-users' computing devices.

The engines described herein may include one or more modules. Modules may include one or more automated agents (e.g., machine learning agents, deep learning agents, etc.). The modules may be trained or built on one or more databases and may be updated periodically and/or continuously.

As used herein, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described herein.

Datastores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores, described herein, can be cloud-based datastores. A cloud-based datastore is a datastore that is compatible with cloud-based computing systems and engines.

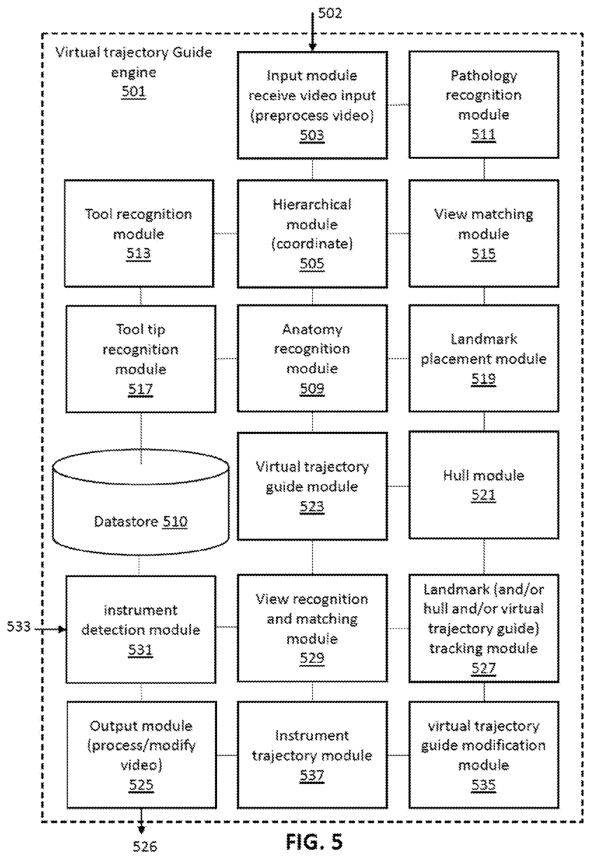

FIG. 5A illustrates one example of a virtual trajectory guide engine 501 that may be included as part of an apparatus as described herein. The virtual trajectory guide engine may be part of a controller and may be locally or remotely accessed. The virtual trajectory guide engine may include an input module 503 for receiving input 502 of one or more videos (e.g., video streams) as described above. The virtual trajectory guide engine may also include multiple different modules and may include a hierarchical module 507 to coordinate the operation of the video trajectory guide engine, including the operation of the modules.

In general, the methods and apparatuses described herein may operate on a video (e.g., video stream). This video may be recorded or, more preferably, may be live from any image-based medical procedure. For example, these procedures may be minimally invasive surgeries (MIS). The video stream may be decomposed into individual frames and may be routed to individual AI agents (“modules”) to perform the various functions described herein. A module may be a deep-learning (DL), machine-learning (ML), or computer vision algorithm (CV) or set of algorithms. For example, these methods and apparatuses may include a hierarchical AI agent (hierarchical module or hierarchical pipeline) 505 to ensure that other modules are activated at the desired time and receive the necessary resources (including information). Other modules may include an anatomy recognition module 509, a pathology module 511, a tool module 513, and view matching module 515. For instance, when the landmarking feature is used in the intraarticular space in the shoulder joint, a pipeline with appropriate anatomy and pathology recognition modules may be activated.

Thus in some examples the apparatus may include a controller, as described herein, which includes one or more processors operating a plurality of modules, such as an anatomy recognition module 509, pathology recognition module 511, and tool recognition module 515 which may operate on the input stream in parallel. The apparatus may also include a tooltip recognition module 517 that operates on the output, i.e., the mask of the probe (e.g., a hook probe), from the tool recognition module 513 and determines the tip of the probe. The landmark placement module 519 may receive surgeon input which triggers the placement of the landmark and may determine the location of the tooltip from the tooltip recognition module 517. The landmark recognition module 519 may also locate the anatomical structure on which the tooltip is being placed by the surgeon from the anatomy recognition module 509 and may determines the outline of the tool from the tool recognition module 513. The landmark recognition module may also extract feature points from the entire frame. Feature points are small visual features, typically arising from subtle textures on anatomical structures. The landmark recognition module may also eliminate feature points generated on the tool, or that are part of the pathology. The outline of the pathology may be obtained from the pathology recognition module 511. The landmark placement module may also eliminate feature points on fast moving objects such as debris and frayed tendons or fibers which may be detected from changes in anatomical structures and pathology between frame to frame. The module may place a landmark after computing its position in the context of the feature points that remain after the deletions described above. The apparatus may register the field of view with the view matching module.

Once the landmark has been placed, a hull module 521 may be activated. The hull module 521 may use the same or a different set of views (e.g., a second set of images). For example the user (e.g., surgeon) may use a probe to identify and in some examples, may trace, a region that includes the landmark. The tool recognition module 513 and/or the tooltip recognition module 517 may be used to determine the tip of the probe and the tip movement by the user may define a set of 3D coordinates that may define a contour including the landmark identified by the landmark placement module 519.

The hull module 521 may then use the contour that is determined from the 3D coordinates of the probe tip to form a virtual hull (virtual 3D hull) that fits the contour identified. The virtual hull may be concave or convex (or both) and intersects with the landmark location. The virtual hull may be displayed on the image(s), or it may be hidden.

A virtual trajectory guide module 523 may then use the virtual hull to generate a virtual trajectory guide. For example, the virtual trajectory guide module 523 may estimate a normal axis to the virtual 3D hull pointing away from the tissue. The virtual trajectory guide module 523 may render the virtual trajectory guide, which (alone or in combination with either or both the virtual hull and/or the landmark) may be shown on the images or any new images in the appropriate region. The virtual trajectory guide module 523 may also modify the shape and/or direction of the virtual trajectory guide based on input from other modules, as described below.

A landmark tracking module 527 (or landmark, hull and/or virtual trajectory guide module) may be activated and it may continuously determine, i.e., track, the position of, the landmark in each subsequent frame in the video stream. In some examples the landmark tracking module 527 may also track the hull and/or the virtual trajectory guide, which may be related thereto. Alternatively a separate hull and/or the virtual trajectory guide tracking module may be used. For each subsequent frame the module may recompute the feature points in the image. The module may also receive the inputs from tool 513, anatomy 509, and pathology 511 recognition modules. As before, feature points on the tool and rapidly moving parts of the frame may be excluded from the set of feature points. The landmark tracking module 527 may then match the feature points and the landmark from the prior frame to the current through a homographic mapping. Once the corresponding feature points have been mapped, the module may infer the position of the landmark relative to the new location of the feature points. The landmark tracking module 527 may check the output from anatomy recognition module 509 to ensure that the landmark stays on the anatomical structure upon which the landmark was initially placed. The system does not require the landmark, hull and/or the virtual trajectory guide to be visible continuously. If the landmark moves off camera, the feature points which are visible are used to track the landmark through the same homographic mapping. The apparatus can optionally either render or suppress the rendering of the landmark, hull and/or the virtual trajectory guide if it/they moves off camera as it is tracked.

The methods and apparatuses described herein may also accommodate situations when the surgeon withdraws the scope and reenters the joint. For example, a view recognition and matching module 529 may be activated. The saved image of the surgical field of view may be recalled and the view matching algorithm may indicate when the scope is approximately in the same position as when the landmark was placed. The ‘view matching’ algorithm may ensure that the landmark, hull and/or the virtual trajectory guide can be reacquired. The view matching algorithm may activate the landmark tracking algorithm and the system may track the landmark, hull and/or the virtual trajectory guide as though there was a temporary occlusion in the field of view. The view recognition and matching module 529 may also be used to indicate when the system is optimally able to place and track landmarks, hulls and/or the virtual trajectory guides.

The view recognition and matching module 529 may be preconfigured with several scenes from specific surgery types where the surgeon is expected to use the landmark tracking feature. When the surgeon navigates to the general site, the view recognition and matching module 529 may indicate a degree of agreement between the field of view and one of the views on which the module was trained. Greater the agreement better the match, the better the tracking performance.

In any of these apparatuses and methods, a tool (e.g., surgical tool, such as an implant, e.g., screw, anchor, etc., cutting/tissue removal tool, cannula, stent, etc.) may be detected using an instrument detection module 531. The instrument detection module 531 may receive indication that a particular procedure being performed or to be performed includes a tool. For example, the instrument detection module 531 may receive input 533 from a user and/or a surgical plan and may determine from the input that a tool is to be used. The instrument detection module may access a data store 510 (e.g., library) to receive information about the shape, contour, size, use parameters, etc. and may identify or detect the instrument within the video. In some variations the instrument detection module 531 may also detect that an instruction that does not match the expected tool is used (e.g., is within the field of view of the video).

The apparatus described herein may also include a virtual trajectory guide modification module 535 that may modify the virtual trajectory guide based on the surgical procedure being or to be performed, and/or the tool(s) (e.g., implant, etc.) to be used at the landmark location. The virtual trajectory guide modification module 535 may modify the virtual trajectory guide, for example, to best suit the particular instrument/tool, which may be determined from the data store (e.g., library) as part of the information about the instruction to be used. For example, the angle of approach onto the tissue for a particular tool may be included in the information from the data store accessed by the virtual trajectory guide modification and may be used to adjust the virtual trajectory guide accordingly.

If the instrument detection module 531 determines that the expected instrument is present, the apparatus may also operate an instrument trajectory module 537 for comparing the actual trajectory of the instrument/tool within the field of view to the virtual trajectory guide. The instrument detection module 531 and/or the instrument trajectory module 537 may determine the actual trajectory of the instrument within the field of view (e.g., relative to the landmark). As mentioned above, if the controller determines that a high level of congruity (e.g., greater than 75 percent congruous) between the virtual trajectory guide and the apparent actual trajectory of the instrument, the instrument trajectory module 537 may output a signal to be displayed at the display, in some examples by modifying the virtual trajectory guide. For example, the display may change a coloration of the pixels representing the virtual trajectory guide indicating that the instrument is properly aligned with the virtual trajectory guide. In a case of low congruity (e.g., less than 75 percent congruous), the module may cause the display to change a coloration of the pixels representing the virtual trajectory guide to indicate that the instrument is not properly aligned with the virtual trajectory guide.

The virtual trajectory guide module 523 may output (via an output module 525) a processed version of the video 526 that has been modified to include the virtual trajectory guide and/or hull and/or landmark as described.

### Examples

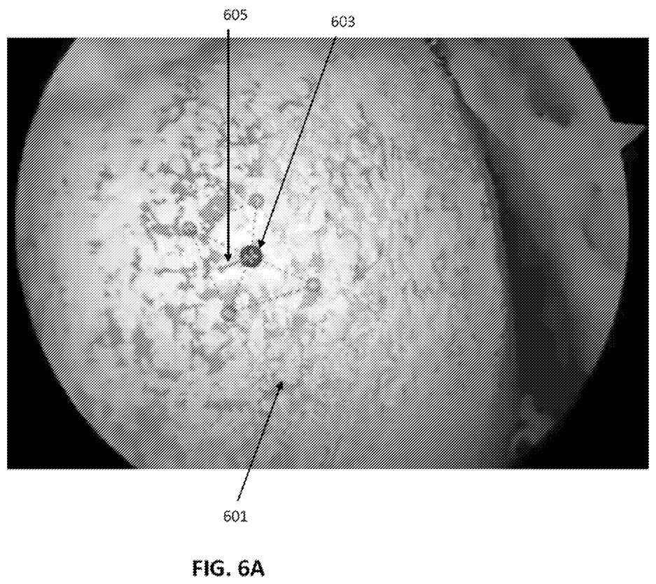

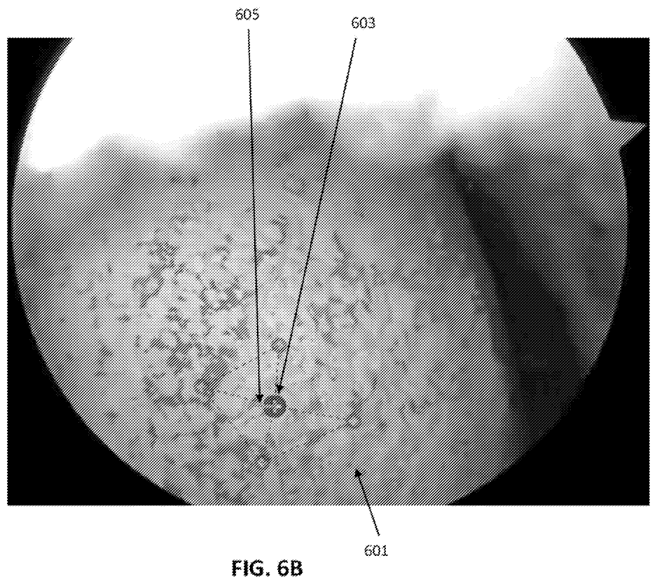

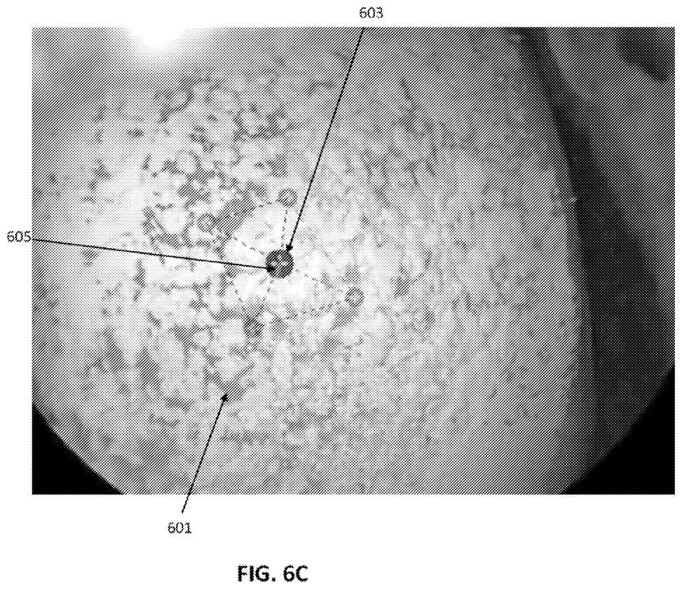

FIGS. 6A-6C illustrate one example of the operation of the methods and apparatus described above. In this example a portion of the anatomy 601 has been tagged with a landmark 603, and a hull contour (not shown) has been determined to fit over a portion of the anatomy including this landmark. A virtual targeting guide 605 extends as a normal from the landmark region of the anatomy. FIGS. 6A-6C show the change in view from as the video is captured from different perspectives. The virtual targeting guide 605 is shown in this example as a vector that extends normal from the surface and maintains its proper normal orientation across the different views of FIGS. 6A-6C.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A computer-implemented method of assisting in a surgical procedure, the method comprising:
receiving a video of a surgical site including an anatomical region;
identifying, from user input, an arbitrary landmark on the anatomical region;
identifying a hull contour conforming to a surface of the anatomical region that includes the arbitrary landmark;
generating three-dimensional (3D) volumetric coordinates for a virtual trajectory guide extending from the hull contour; and
outputting a modified video of the surgical site including the virtual trajectory guide, wherein an orientation of the virtual trajectory guide relative to the anatomical region is maintained as a field of view of the surgical site changes during the modified video.

2. The method of claim 1, wherein receiving the video of the surgical site comprises capturing the video.

3. The method of claim 1, wherein identifying the arbitrary landmark comprises extracting feature points from the anatomical region near the arbitrary landmark, further comprising excluding feature points that are on debris or rapidly-changing structures.

4. The method of claim 1, wherein identifying the hull contour comprises identifying the hull contour based on a distal tip region of a probe moved over the anatomical region.

5. The method of claim 4, further comprising identifying a distal tip of the probe from the video and extracting 3D coordinates of the distal tip as it moves over the anatomical region.

6. The method of claim 1, wherein generating the 3D volumetric coordinates for the virtual trajectory guide comprises generating the 3D volumetric coordinates so that the virtual trajectory guide passes through the arbitrary landmark.

7. The method of claim 1, wherein the virtual trajectory guide comprises a vector.

8. The method of claim 1, wherein the virtual trajectory guide comprises a pipe.

9. The method of claim 1, further comprising modifying the video of the surgical site to include the virtual trajectory guide, wherein the orientation of the virtual trajectory guide relative to the anatomical region is maintained as the field of view of the surgical site changes.

10. The method of claim 9, wherein modifying the video further comprises modifying the video to show the hull contour.

11. The method of claim 9, wherein modifying the video further comprises modifying the video to show the arbitrary landmark.

12. The method of claim 1, wherein outputting the modified video is performed in real time.

13. The method of claim 1, further comprising identifying an instrument within the field of view of the video and comparing a trajectory of the instrument to the virtual trajectory guide.

14. The method of claim 13, further comprising modifying the modified video to indicate that the trajectory of the instrument is congruent with the virtual trajectory guide above a threshold for congruence.

15. The method of claim 14, wherein the threshold for congruence is 75% or greater.

16. The method of claim 14, wherein modifying the video comprises changing an output parameter of the virtual trajectory guide.

17. The method of claim 13, wherein identifying the instrument within the field of view of the video comprises one or both of receiving user input of the instrument to be used or accessing a surgical plan to determining if the instrument is to be used.

18. The method of claim 1, further comprising modifying the virtual trajectory guide based on an instrument to be used during the surgical procedure.

19. A computer-implemented method of assisting in a surgical procedure, the method comprising:
receiving a video of a surgical site including an anatomical region;
identifying, from user input, an arbitrary landmark on the anatomical region;
identifying a hull contour conforming to a surface of the anatomical region that includes the arbitrary landmark based on a distal tip region of a probe moved over the anatomical region;
generating three-dimensional (3D) volumetric coordinates for a virtual trajectory guide extending from the hull contour using an axis normal to the hull contour; and
outputting a modified video of the surgical site including the virtual trajectory guide, wherein the orientation of the virtual trajectory guide relative to the anatomical region is maintained as a field of view of the surgical site changes during the modified video.

20. A non-transitory computer-readable medium including contents that are configured to cause one or more processors to perform a method comprising:
receiving a video of a surgical site including an anatomical region;
identifying, from user input, an arbitrary landmark on the anatomical region;
identifying a hull contour conforming to a surface of the anatomical region that includes the arbitrary landmark;
generating three-dimensional (3D) volumetric coordinates for a virtual trajectory guide extending from the hull contour; and
outputting a modified video of the surgical site including the virtual trajectory guide, wherein an orientation of the virtual trajectory guide relative to the anatomical region is maintained as a field of view of the surgical site changes during the modified video.

21.-55. (canceled)
